(19) United States
(12) Patent Application Publication
(10) Pub. No.: US 2014/0030201 A1
(43) Pub. Date: Jan. 30, 2014

(54) A SKIN LIGHTENING COMPOSITION

(71) Applicants: Rebecca Susan Ginger, Sharnbrook (GB); Melanie Jane Sandel, Newbridge (GB)

(72) Inventors: Rebecca Susan Ginger, Sharnbrook (GB); Melanie Jane Sandel, Newbridge (GB)

(21) Appl. No.: 13/994,138

(22) PCT Filed: Dec. 21, 2010
(86) PCT No.: PCT/EP2011/068709

§ 371 Date: Oct. 14, 2013

(30) Foreign Application Priority Data
Dec. 21, 2010 (EP) 10196303.1

(51) Int. Cl. A61K 8/49; A61K 8/46

(52) U.S. Cl. CPC A61K 8/4973; A61K 8/46
USPC 424/62

(57) ABSTRACT

This invention relates to a composition comprising a synergistic combination of ketoconazole and sulforaphane for use in skin lightening. This invention is based on the fact that a combination of ketoconazole and sulforaphane has been found to synergistically inhibit pigment production in B16 monolayer cultures. Thus the composition, when applied topically or imbibed over an appropriate length of time in-vivo, would be expected to cause skin lightening, or to reduce blemishes and/or hyperpigmented spots and/or solar lentigines leading to an improvement in skin tone and evenness.

Desired skin colour is a major unmet consumer need in Asia. Consumers particularly desire even skin colour, absence of age spots (solar lentigines) and lighter overall skin tone. One solution is to use biological actives that reduce the activity of melanocytes in skin. These cells, present in the basal layer of the epidermis, make the darkly coloured pigment melanin and export it in small export vesicles called melanosomes to the neighbouring keratinocytes. It is well described in the literature that compounds which reduce melanin synthesis when topically applied to the skin will reduce skin darkness over time.

This invention relates to a composition comprising a synergistic combination of ketoconazole and sulforaphane for use in skin lightening. This invention is based on the fact that a combination of ketoconazole and sulforaphane has been found to synergistically inhibit pigment production in B16 monolayer cultures. Thus the composition, when applied topically or imbibed over an appropriate length of time in-vivo, would be expected to cause skin lightening, or to reduce blemishes and/or hyperpigmented spots and/or solar lentigines leading to an improvement in skin tone and evenness.

Ketoconazole is sold (for example as Nizoral™ by Johnson & Johnson Inc. and Daktarin™ Gold by Janssen Pharmaceutica NV) in a topical and oral over-the-counter (OTC) preparation for the treatment of fungal diseases.

WO 2007/072216 A2 discloses a therapeutic kit including a therapeutic azole with increased solubility. The kit includes an aerosol packaging assembly having a container accommodating a pressurized product and an outlet capable of releasing the pressurized product as a foam. The pressurized product is a foamable composition including, amongst other things, a therapeutic azole. In exemplary embodiments, the therapeutic azole is an imidazole or triazole selected from a group including ketoconazole. In one or more embodiments, the foamable composition further includes at least one additional therapeutic agent selected from the group including a skin whitening agent.

In WO 02/053108 A2, retinoic acid is disclosed as having been employed to treat a variety of skin conditions including age spots and discoloration. Compounds which inhibit acyl coenzyme A (CoA): retinol acyl transferase/lecithin retinol acyl transferase (ARAT/LRAT), retinal reductase, cellular retinoic acid binding protein II (CRAB PII) and retinoic acid oxidation (the latter catalyzed by cytochrome P450 systems), and others which enhance retinol dehydrogenase are collectively termed “boosters”. The boosters, alone or in combination with each other, potentiate the action of a retinoid by increasing the amount of retinol available for conversion to retinoic acid and inhibiting the degradation of retinoic acid. Ketoconazole is identified as a retinoic acid oxidation inhibitor.

In Mun et al (Biol. Pharm. Bull., 27(6), 806-809 (2004)), the inhibitory effect of miconazole is described on melanogenesis in B16 melanoma cells. Tyrosinase activity and melanin content were dose dependently decreased by the azole as compared with untreated cells, however this is accompanied by decreased cellular viability.

### SUMMARY OF THE INVENTION

In a first aspect of the invention, a skin lightening composition is provided, the composition comprising ketoconazole and sulforaphane. Ketoconazole has the following structure:

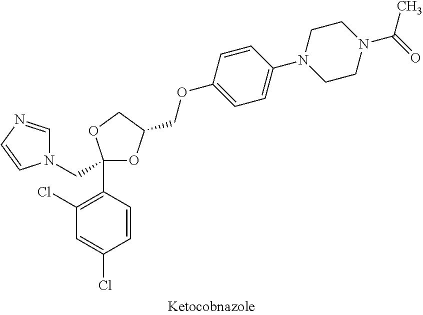 [SMILES: CC(=O)N1CCN(c2ccc(OC[C@H]3CO[C@](Cn4ccnc4)(c4ccc(Cl)cc4Cl)O3)cc2)CC1]

and sulforphane has the following structure:

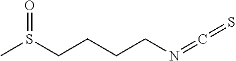

It is obtained from cruciferous vegetables such as broccoli. The enzyme myrosinase transforms glucoraphanin, a glucosinolate, into sulforaphane upon damage to the plant (such as from chewing). Young sprouts of broccoli and cauliflower are particularly rich in glucoraphanin.

The skin lightening composition may comprise 0.001 to 2, preferably 0.005 to 0.5% w/w ketoconazole. The skin lightening composition may comprise 0.001 to 2, preferably 0.01 to 1% w/w sulforaphane.

The sulforaphane may be in the form of the L-isomer, preferably exclusively in the form of the L-isomer.

The skin lightening composition may be in the form of an oral or topical composition.

In a second aspect of the invention, the composition of the first aspect is provided for use in skin lightening. In one embodiment of this aspect, the composition of the first aspect is provided for use in skin lightening, wherein the composition is used such that the daily dosage for oral use of ketoconazole is 50 to 200, preferably 50 to 100 mg; and the daily dosage for oral use of sulforaphane is 50 to 600, preferably 200 to 400 mg.

In the alternative, use of ketoconazole and sulforaphane is provided in the manufacture of the composition of the first aspect for lightening skin. In a further embodiment of this alternative, use of ketoconazole and sulforaphane is provided in the manufacture of the composition of the first aspect for lightening skin, wherein the composition is administered such that the daily dosage for oral use of ketoconazole is 50 to 200, preferably 50 to 100 mg; and the daily dosage for oral use of sulforaphane is 50 to 600, preferably 200 to 400 mg.

In a further alternative, a method of lightening the skin of a human is provided, the method comprising the step of a person in need thereof imbibing the composition of the first aspect. In a further embodiment of this further alternative, a method of lightening the skin of a human is provided, the method comprising the step of a person in need thereof imbibing the composition of the first aspect such that the daily dosage of ketoconazole is 50 to 200, preferably 50 to 100 mg; and the daily dosage of sulforaphane is 50 to 600, preferably 200 to 400 mg.

### SUMMARY OF THE FIGURES

The invention is illustrated with reference to the following figures wherein:

FIG. 1 shows darkly pigmented MelanoDerm™ cultures treated with 1 μM ketoconazole for 14 days (right hand side) versus DMSO control (left hand side) with melanin content visualised with Masson-Fontana staining;

FIG. 2 shows darkly pigmented MelanoDerm™ cultures treated with 1 μM ketoconazole for 14 days (righthand side) versus DMSO control (left hand side) with melanocytes visualised with MART-1 immuno-staining; and

FIG. 3 shows light microscopy images of normal human melanocytes derived from a dark skinned donor after treatment with 1 μM ketoconazole for 5 days (right hand side) versus DMSO control (left hand side).

### DETAILED DESCRIPTION OF THE INVENTION

Topical Compositions

It should be known that commercially acceptable and conventional vehicles may be used in topical compositions of the invention, acting as diluents, dispersants and/or carriers for the skin lightening agents described herein and for any other optional but often preferred ingredients. Therefore, cosmetically acceptable vehicle suitable for use in this invention may be aqueous-based, anhydrous or an emulsion whereby a water-in-oil or oil-in-water emulsion is generally preferred. If the use of water is desired, water typically makes up the balance of the composition, and preferably makes up from about 5 to about 99%, and most preferably from about 40 to about 80% by weight of the topical composition, including all ranges subsumed therein.

In addition to water, organic solvents may be optionally included to act as carriers or to assist carriers within the compositions of the present invention. Illustrative and non-limiting examples of the types of organic solvents suitable for use in the present invention include alkanols like ethyl and isopropyl alcohol, mixtures thereof or the like.

Other optional additives suitable for use include ester oils like isopropyl myristate, cetyl myristate, 2-octyldodecyl myristate, avocado oil, almond oil, olive oil, neopentylglycol dicaprate, mixtures thereof or the like. Typically, such ester oils assist in emulsifying the composition of this invention, and an effective amount is often used to yield a stable, and most preferably, water-in-oil emulsion.

Emollients may also be used, if desired, as carriers within the composition of the present invention. Alcohols like 1-hexadecanol (i.e. cetyl alcohol) are often desired as are the emollients generally classified as silicone oils and synthetic esters. Silicone oils suitable for use include cyclic or linear polydimethylsiloxanes containing from 3 to 9, preferably from 4 to 5, silicon atoms. Non-volatile silicone oils useful as an emollient material in the inventive composition described herein include polyalkyl siloxanes, polyalkylaryl siloxanes and polyether siloxane copolymers. The essentially non-volatile polyalkyl siloxanes useful herein include, for example, polydimethylsiloxanes.

The ester emollients that may optionally be used are:
(1) Alkenyl or alkyl esters of fatty acids having 10 to 20 carbon atoms. Examples thereof include isoarachidyl neopentanoate, isononyl isonanonoate, oleyl myristate, oleyl stearate, and oleyl oleate.
(2) Ether-esters such as fatty acid esters of ethoxylated fatty alcohols.
(3) Polyhydric alcohol esters. Ethylene glycol mono and di-fatty acid esters, diethylene glycol mono- and di-fatty acid esters, polyethylene glycol (200-6000) mono- and di-fatty acid esters, propylene glycol mono- and di-fatty acid esters, polypropylene glycol 2000 monooleate, polypropylene glycol 2000 monostearate, ethoxylated propylene glycol monostearate, glyceryl mono- and di-fatty acid esters, polyglycerol poly-fatty esters, ethoxylated glyceryl mono-stearate, 1,3-butylene glycol monostearate, 1,3-butylene glycol distearate, polyoxyethylene polyol fatty acid ester, sorbitan fatty acid esters, and polyoxyethylene sorbitan fatty acid esters are satisfactory polyhydric alcohol esters.
(4) Wax esters such as beeswax, spermaceti, stearyl stearate and arachidyl behenate.
(5) Sterols esters, of which cholesterol fatty acid esters are examples.

Emollients when used, typically make up from about 0.1 to about 50% by weight of the composition, including all ranges subsumed therein.

Fatty acids having from 10 to 30 carbon atoms may also be included as acceptable carriers within the composition of the present invention. Illustrative examples of such fatty acids include pelargonic, lauric, myristic, palmitic, stearic, isostearic, oleic, linoleic, arachidic, behenic or erucic acid, and mixtures thereof. Compounds that are believed to enhance skin penetration, like dimethyl sulfoxide, may also be used as an optional carrier.

Humectants of the polyhydric alcohol type may also be employed in the compositions of this invention. The humectant often aids in increasing the effectiveness of the emollient, reduces scaling, stimulates removal of built-up scale and improves skin feel. Typical polyhydric alcohols include glycerol, polyalkylene glycols and more preferably alkylene polyols and their derivatives, including propylene glycol, dipropylene glycol, polypropylene glycol, polyethylene glycol and derivatives thereof, sorbitol, hydroxypropyl sorbitol, hexylene glycol, 1,3-butylene glycol, 1,2,6-hexanetriol, ethoxylated glycerol, propoxylated glycerol and mixtures thereof. For best results the humectant is preferably propylene glycol or sodium hyaluronate. The amount of humectant may range anywhere from 0.2 to 25%, and preferably, from about 0.5 to about 15% by weight of the composition, based on total weight of the composition and including all ranges subsumed therein.

Thickeners may also be utilized as part of the acceptable carrier in the compositions of the present invention. Typical thickeners include cross-linked acrylates (e.g. Carbopol 982), hydrophobically-modified acrylates (e.g. Carbopol 1382), cellulosic derivatives and natural gums. Among useful cellulosic derivatives are sodium carboxymethylcellulose, hydroxypropyl methylcellulose, hydroxypropyl cellulose, hydroxyethyl cellulose, ethyl cellulose and hydroxymethyl cellulose. Natural gums suitable for the present invention include guar, xanthan, sclerotium, carrageenan, pectin and combinations of these gums. Amounts of the thickener may range from 0.0 to 5%, usually from 0.001 to 1%, optimally from 0.01 to 0.5% by weight of the composition.

Collectively the water, solvents, silicones, esters, fatty acids, humectants and/or thickeners will constitute the acceptable carrier in amounts from 1 to 99.9%, preferably from 80 to 99% by weight of the composition.

Surfactants may also be present in compositions of the present invention. Total concentration of the surfactant will range from about 0 to about 40%, and preferably from about 0 to about 20%, optimally from about 0 to about 5% by weight of the composition. The surfactant may be selected from the group consisting of anionic, nonionic, cationic and amphoteric actives. Particularly preferred nonionic surfactants are those with a C10-C20 fatty alcohol or acid hydrophobe condensed with from 2 to 100 moles of ethylene oxide or propylene oxide per mole of hydrophobe; mono- and di-fatty acid esters of ethylene glycol; fatty acid monoglyceride; sorbitan, mono- and di-C8-C20 fatty acids; block copolymers (ethylene oxide/propylene oxide); and polyoxyethylene sorbitan as well as combinations thereof. Alkyl polyglycosides and saccharide fatty amides (e.g. methyl gluconamides) are also suitable nonionic surfactants.

Preferred anionic surfactants include soap, alkyl ether sulfate and sulfonates, alkyl sulfates and sulfonates, alkylbenzene sulfonates, alkyl and dialkyl sulfosuccinates, C8-C20 acyl isethionates, acyl glutamates, C8-C20 alkyl ether phosphates and combinations thereof.

Perfumes may be used in the composition of this invention. Illustrative non-limiting examples of the types of perfumes that may be used include those comprising terpenes and terpene derivatives like those described in Bauer, K., et al., Common Fragrance and Flavor Materials, VCH Publishers (1990).

Illustrative yet non-limiting examples of the types of fragrances that may be used in this invention include myrcene, dihydromyrenol, citral, tagetone, cis-geranic acid, citronellic acid, mixtures thereof or the like.

Preferably, the amount of fragrance employed in the composition of this invention is in the range from about 0.0% to about 10%, more preferably about 0.00001% to about 5 wt %, most preferably about 0.0001% to about 2% by weight of the compound.

Various types of optional additional active ingredients may be used in the compositions of the present invention. Actives are defined as skin benefit agents other than emollients and other than ingredients that merely improve the physical characteristics of the composition. Although not limited to this category, general examples include talcs and silicas, as well as alpha-hydroxy acids, beta-hydroxy acids, zinc salts, and sunscreens.

Beta-hydroxy acids include salicylic acid, for example. Zinc pyrithione is an example of the zinc salts useful in the composition of the present invention.

Sunscreens include those materials commonly employed to block ultraviolet light. Illustrative compounds are the derivatives of PABA, cinnamate and salicylate. For example, avobenzophenone (Parsol 1789®) octyl methoxycinnamate and 2-hydroxy-4-methoxy benzophenone (also known as oxybenzone) can be used. Octyl methoxycinnamate and 2-hydroxy-4-methoxy benzophenone are commercially available under the trademarks, Parsol MCX and Benzophenone-3, respectively. The exact amount of sunscreen employed in the compositions can vary depending upon the degree of protection desired from the sun's UV radiation. Additives that reflect or scatter the suns rays may also be employed. These additives include oxides like zinc oxide and titanium dioxide.

Many compositions, especially those containing water, should be protected against the growth of potentially harmful microorganisms. Anti-microbial compounds, such as triclosan, and preservatives are, therefore, typically necessary. Suitable preservatives include alkyl esters of p-hydroxybenzoic acid, hydantoin derivatives, propionate salts, and a variety of quaternary ammonium compounds. Particularly preferred preservatives of this invention are methyl paraben, propyl paraben, phenoxyethanol and benzyl alcohol. Preservatives will usually be employed in amounts ranging from about 0.1% to 2% by weight of the composition.

Still other optional ingredients that may be used with the composition of this invention include dioic acids (e.g. malonic acid and sebacic acid), antioxidants like vitamin E, retinoids, including retinoic acid, retinal, retinol and retinyl esters, conjugated linoleic acid, petroselinic acid and mixtures thereof, as well as any other conventional ingredients well known for wrinkle-reducing, anti-acne effects and reducing the impact of sebum.

When making the composition of the present invention, the desired ingredients are mixed in no particular order and usually at temperatures from about 70 to about 80° C. and under atmospheric pressure.

The packaging for the composition of this invention can be a patch, bottle, tube, roll-ball applicator, propellant driven aerosol device, squeeze container or lidded jar.

Oral Compositions

Oral compositions of the invention may be in the form of capsules, pills, tablets, granules, solutions, suspensions or emulsions.

If the composition is water-based, i.e. comprises at least 70% w/w water, it has the sensation of being a regular water-based product and would thus be consumed on a regular basis as part of a consumer's normal diet. For example it could replace a fruit juice normally consumed at breakfast. Preferably the water-based composition has a viscosity of from 2 to 100 centipoise at a shear rate of 1 s-1 and at 25 degrees centigrade.

The composition may comprise from 0.2 to 10%, preferably from 0.4 to 5& w/w oil. The oil may comprise at least 12, preferably at least 20% w/w eicosapentaenoic acid (EPA) and docosahexaenoic acid (DHA), both omega 3 polyunsaturated acids and known as fish oils. Increased intake of EPA has been shown to be beneficial in coronary heart disease, high blood pressure, and inflammatory disorders such as rheumatoid arthritis.

Antioxidant is required in order to prevent or slow down the natural oxidative degradation of the fish oil. Suitable antioxidants can be selected, although not exclusively, from the following list, either singularly or in combination: tert-butylhydroquinone (TBHQ), ascorbyl esters such as ascorbyl palmitate, ascorbic acid, tocopherols, rosemary extract, fruit concentrates or extracts, black or green tea extract, propyl gallate, essential oils or oleoresins, butylated hydroxyanisole (BHA), butylated hydroxytoluene (BHT), citric acid or esters, coenzyme Q10, tocotrienols, polyphenols, phenolic compounds and flavonoids.

Especially preferred antioxidants are vitamins C and E. Not only are these effective antioxidants but they also have been shown to give skin benefits when consumed.

The amount of antioxidant should be added sufficient to prevent the fish oil from going rancid over a typical shelf-life of 6 months. Clearly the amount of antioxidant will depend on the type and activity of the antioxidant used. However, preferably the product has a ratio of antioxidant to oil of from 1:10 to 1:100 based on the antioxidant activity of vitamin C. For these purposes an antioxidant activity is as measured using an appropriate assay, for example Trolox equivalent antioxidant capacity.

At least 0.01%, preferably from 0.05 to 3%, more preferably from 0.1 to 1% w/w phospholipid emulsifier, such as lecithin, has been found to be suitable for emulsifying high quantities of fish oil.

The composition may also comprise from 0.01 to 0.5%, preferably from 0.01 to 0.3% w/w soy isoflavones. Soy isoflavones are components within soy that have a biological function similar to oestrogen, including the promotion of dermal matrix protein synthesis. In addition, they have also been shown to have anti-inflammatory properties and stimulate synthesis of hyaluronic acid, a proteoglycan in skin which can retain water and thereby influence skin firmness. Preferably the soy isoflavones are selected from genistein and daidzein.

The composition may also comprise from 0.0005 to 0.1%, preferably from 0.002 to 0.04% w/w carotenoids. The carotenoids, being oil soluble, would be comprised predominantly within the oil phase. Highly preferred carotenoids are beta-carotene, and lycopene. These carotenoids provide moderate protection from UV induced erythema, thought to be due to their antioxidant functionality including scavenging of reactive oxygen species.

The composition is prepared from separate aqueous and oil phases. In general the water-soluble ingredients are combined to form the aqueous phase and the oil-soluble ingredients combined to form the oil phase. Then the two phases are blended together to form a homogenous stable emulsion. The stable emulsion may then be packaged in a sealed container such as a metal, coated cardboard, for example as marketed by Tetra Pak™, or a plastics container. The container is then preferably sealed so as to give no headspace or a gas filled, for example nitrogen or carbon dioxide, headspace. This assists still further in preventing the fish oil oxidising. Alternatively the emulsion may be frozen and packaged and sold as a frozen consumer product.

In-Vitro Studies of the Skin Lightening Effect of Ketoconazole

Materials

MelanoDerm™ Cultures

MatTek MelanoDerm™ cultures (a pigmented 3D-Living Skin Equivalent (LSE) model) purchased from the MatTek Corporation. Dark cultures (MEL-300-B), for evaluation of skin lightening potential, were prepared in a long life maintenance medium (EPI-100-LLMM available from the MatTek Corporation) for good pigment production whilst preserving acceptable histology.

On arrival samples were replaced into growth medium and left at 37° C. for 2 hours to recover prior to the administration of test items. Test actives were applied to the samples by addition to the growth medium. Fresh growth medium containing dimethyl sulphoxide (DMSO) alone or test actives was replaced every 2-3 days and the cultures were grown in this way for 14 days prior to harvesting and analysis. Prior to extraction or fixation cultures were assayed using the WST-1 cell proliferation assay.

B16 Monolayer Cultures

B16 mouse melanoma cells were cultured in Eagle's minimal essential medium (EMEM) supplemented with 10% foetal calf serum (FCS) and 2 mM L-glutamine at 37° C., 5% CO2 in T175 tissue culture flasks and were sub-cultured twice weekly using trypsin in EDTA. For assay, 25 000 cells per well in 500 μl volume were seeded to a 48 well plate and adhered overnight. The following morning standard culture media was replaced by EMEM, 10% FCS, 4 mM L-glutamine (500 μl) and test actives added. Cells were then incubated for 72 hours. At 72 after which the supernatants were removed and analysed for melanin content by spectrophometry at 450 nm against a reference standard curve. The B16 cell monolayer was dissolved in Triton™/PBS at 4° C. for 20 minutes, centrifuged at 13 000 rpm for 5 minutes at 4° C. and analysed for total protein content using a standard bicinchoninic acid assay (BCA) assay against a reference standard curve. Melanin results were normalised to total protein.

Normal Human Melanocytes

Primary human melanocytes were obtained from Invitrogen Cascade Biologics (HEMn-DP) and grown in Cascade melanocyte media (M-254CF-500+human melanocyte growth supplement (HMGS) supplement containing phorbol myristate acetate (PMA)) according to the manufacturer's instructions. Once established, cultures were routinely grown in T175 tissue culture flasks and could generally be passaged for use up to six (P6) or seven (P7) times. Once the cells reached about 70% confluence they were treated with trypsin, spun down and re-suspended in an appropriate volume of media ready for plating out. Cells seeded at 2×10⁵ cells per well in a 6-well plate and incubated in 2 ml medium per well. Cells were allowed 16 hours to settle on the cover-slips before addition of treatments. Cells were treated for 5 days with DMSO vehicle or test actives dissolved in DMSO and light microscope images captured.

Azoles

Ketoconazole, fluconazole, imidazole, oxazole and thiazole were obtained from Sigma Aldrich Company Limited.

Methods

Melanin Quantitation

Melanin content of the cultures was determined following the recommended by MatTek (solvable extraction protocol) using a proprietary solvent known as Solvable™ (available from Perkin-Elmer) as the extraction solvent. Protein was extracted as described previously and quantified using the BCA assay.

Histological Analysis

Post treatment and WST-1 analysis cultures were fixed in neutral buffered formalin (NBF) for 4 hours and processed for histology to provide formalin fixed paraffin embedded (FFPE) tissue blocks. Each culture was bisected and embedded in the same paraffin embedded block so that when sectioned, one section provided two non-serial sections. FFPE tissue was cut to provide four slides per block, each slide containing two non-serial sections per slide. Sets of slides were subject to immuno-staining with an antibody against the melanocyte marker MART-1 (Abcam Plc), or stained with Masson Fontana and representative images captured.

Results

Melanogenesis in B16 Monolayer Cultures

Each test active was applied in DMSO to n=3 cultures. The results are summarised in table 1.

TABLE 1
Normalised melanin (per mg protein) from B16 monolayer cultures
(the first DMSO control relates to the ketoconazole results only and
the second DMSO control relates to the remaining results).

| Treatment | Normalised melanin (μg/mg protein) |
|---|---|
| DMSO control | 0.186 ± 0.021 |
| 5 μM ketoconazole | 0.173 ± 0.032 |
| 20 μM ketoconazole | 0.004 ± 0.001 |
| 50 μM ketoconazole | 0.007 ± 0.003 |
| DMSO control | 0.187 |
| | 0.188 |
| | 0.172 |
| 20 μM flucoazole | 0.232 |
| | 0.214 |
| | 0.229 |
| 20 μM imidazole | 0.180 |
| | 0.231 |
| | 0.240 |
| 20 μM oxazole | 0.217 |
| | 0.210 |
| | 0.185 |
| 20 μM thiazole | 0.228 |
| | 0.255 |
| | 0.236 |

Melanogenesis in MelanoDerm™ Cultures

Ketoconazole was applied in DMSO to n=6 cultures. Three cultures from each treatment group were extracted for melanin and protein quantitation and three were fixed, wax embedded, sectioned and stained to visualise histological integrity (H&E) and melanin (Fontana-Masson). The results for normalised melanin (per mg protein) are summarised in table 2.

TABLE 2
Normalised melanin (per mg protein) from MelanoDerm ™ cultures.

| Treatment | Normalised melanin (μg/mg protein) |
|---|---|
| DMSO control | 74.560 |
| | 103.845 |
| | 90.333 |
| 1 μM ketoconazole | 59.276 |
| | 57.292 |
| | 58.573 |

The total extracted protein concentration was relatively consistent for all treatments (data not shown) suggesting that there were not any substantial cytotoxic effects on the cultures.

Histological Analysis

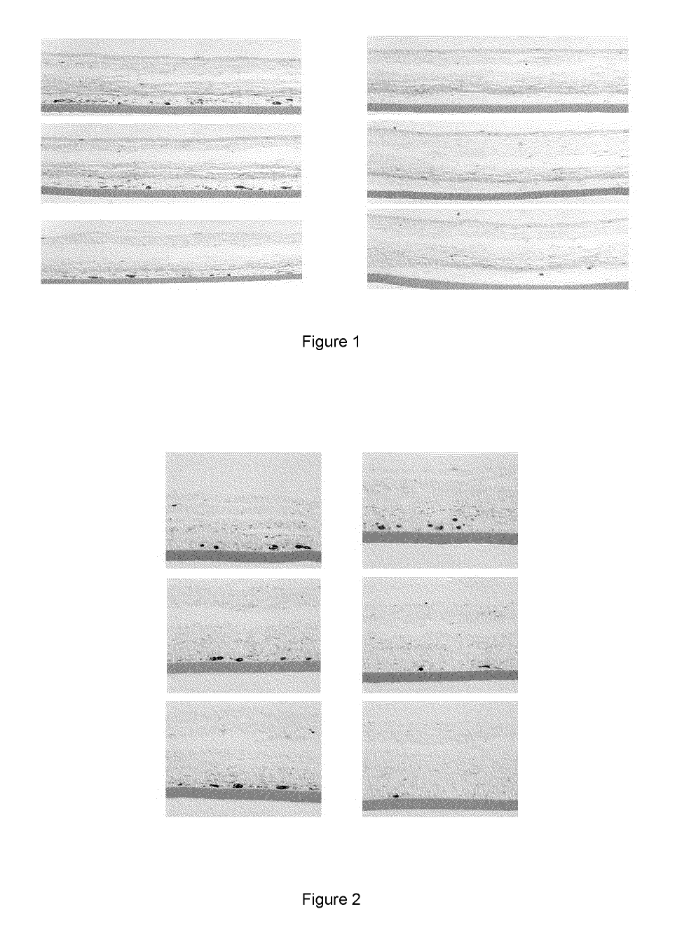

FIG. 1 shows that the normalised melanin extracted from the culture treated with 1 μM ketoconazole consistently reduced the levels of extractable melanin and had a marked effect on the melanin content of the basal melanocytes as shown after Masson-Fontana staining. FIG. 2 immuno-staining with an antibody against the melanocyte marker MART-1 shows that the absence of Masson-Fontana stained melanin in FIG. 1 was not due to the elimination of melanocytes from these cultures.

Evaluation of Ketoconazole on Growth and Morphology of Normal Human Melanocytes (NHM) in Monolayer Culture

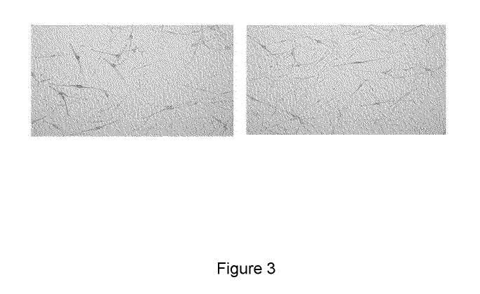

NHMs grown in monolayer cultures were treated with 1 μM ketoconazole for 5 days and assessed by light microscopy. The results shown in FIG. 3 indicate that there was no evidence that treatment had an adverse effect on NHM morphology or proliferation. In particular it can be seen that the proportion of bipolar cells (normal cells) for the NHMs treated with ketoconazole is practically identical to the control.

Conclusions

Ketoconazole very effectively inhibits melanin production in B16 monolayer cultures and MelanoDerm™ cultures at 1 μM. Furthermore there is no detectable effect on the viability of either monolayer human melanocytes or epidermal cultures at this dose. Surprisingly other azoles and in particular fluconazole, imidazole, oxazole and thiazole did not effectively inhibit melanin production in B16 monolayer cultures.

In-Vitro Delivery of Ketoconazole into Human Skin

Method

This study quantified the in-vitro human skin penetration after application of a cream formulation containing 2% (w/w) ketoconazole (Daktarin™ Gold). Eight diffusion cells were prepared (using female abdominal skin from three donors) plus three undosed control cells (for assay validation purposes). Epidermal membranes were used and integrity assessed by measuring electrical resistance. Permeation of active was measured over 24 hours following application of a target 5 mg/cm2 dose. Suitable liquid chromatography analytical assays were developed, with active peaks separated from formulation, skin and tape strip derived peaks.

Distribution within the skin at 24 hours was determined by measuring the levels of active within the stratum corneum (tape strips) and the epidermis plus any remaining lower stratum corneum (following tape stripping). The skin was wiped prior to tape stripping to remove active remaining at the surface. Levels in the donor chamber sealing grease and on the donor chambers were also measured (donor chamber wash).

Results

The results are presented in table 3 below.

TABLE 3
Recovered ketoconazole from in-vitro delivery into human skin
after 24 hours (n = 8).

| Compartment | Ketoconazole (% recovered) |
|---|---|
| Surface of skin | 83.6 ± 2.4 |
| Donor chamber | 4.00 ± 1.26 |
| Strip 1 | 4.92 ± 2.03 |
| Strips 2-3 | 0.858 ± 0.255 |
| Strips 4-6 | 0.318 ± 0.062 |
| Strips 7-10 | 0.192 ± 0.046 |
| Epidermis† | 2.02 ± 0.74 |
| Permeation chamber | 3.49 ± 0.26 |
| Overall recovery | 99.4 ± 1.2 |

†Will also include any remaining stratum corneum remaining following tape stripping

Conclusion

Ketoconazole showed low but adequate delivery into human skin.

Combination of Ketoconazole with Sulforaphane

Method

B16 F10 cells (mouse melanoma cell line) were obtained from ATCC and grown in Lonza Biowhittaker BE12-662F media. Trypsin in ethylenediaminetetraacetic acid (EDTA) (Sigma T3924) and Dulbecco's Phosphate Buffered Saline (DPBS) (Sigma D8537) were used to split the cells. Plates were set up at a concentration of 2.5×10⁴ cells per well in 48 well plates in Phenol Red free media (Sigma D1145).

After overnight incubation, media was removed and media with sulforaphane (available from Sigma-Aldrich Company Limited as at least 95% L-sulforaphane) in DMSO and ketoconazole in DMSO solutions were added. 500 μl of the media and solutions were added to 4 wells of a 48 well plate and the plates incubated at 37° C. for a further 3 days. Thereafter the melanin concentration and cell numbers were measured.

Melanin concentration was measured by making up standards each day and pipetting 100 μl of each standard in duplicate onto Greiner plates. For the blank plate Phenol Red free media was used. 100 μl from each well was also pipetted in duplicate onto Greiner plates and the plates read on a Dynex plate reader at 450 nm.

Cell numbers were measured by washing the wells twice with phosphated buffer solution (PBS). Then 1 ml of trypsin EDTA diluted in PBS at 1:10 v/v was added to each well and the wells observed for cell detachment. All media from each well was then collected the media assayed by adding 100 μl of the media to 9.9 ml of Coulter Isoton II diluent. Cell numbers were assessed using the Z1 Coulter Particle Counter.

Results

The results are summarised in table 5.

TABLE 5
Melanin (μg) per cell.

| | Average | |
|---|---|---|
| | μg melanin/ | |
| | cell | |
| | (4 tests) | % vehicle control |
| 10 μM L-sulforaphane | 0.00026453 | 86.88710608 |
| 1 μM ketoconazole | 0.00029155 | 95.76492704 |
| 10 μM L-sulforaphane + | 6.8138E−05 | 22.38093578 |
| 1 μM ketoconazole | | |
| Media control | 0.00030445 | 100 |
| DMSO control | 0.00028774 | 94.51056836 |

Conclusion

Significant synergy in reducing melanin content per cell is exhibited in this in-vitro assay by combining L-sulforaphane with ketoconazole.

1. A skin lightening composition comprising ketoconazole and sulforaphane.

2. A skin lightening composition according to claim 1 comprising 0.001 to 2, preferably 0.005 to 0.5 w/w ketoconazole.

3. A skin lightening composition according to claim 1 comprising 0.001 to 2, preferably 0.01 to 1% w/w sulforaphane.

4. A skin lightening composition according to claim 1, wherein the sulforaphane is in the form of the L-isomer, preferably exclusively in the form of the L-isomer.

5. A skin lightening composition according to claim 1 in the form of an oral or topical composition.

6. A composition according to claim 1 for use in skin lightening, wherein the composition is used such that the daily dosage for oral use of ketoconazole is 50 to 200, preferably 50 to 100 mg; and the daily dosage for oral use of sulforaphane is 50 to 600, preferably 200 to 400 mg.

7. Use of ketoconazole and sulforaphane in the manufacture of a composition according to claim 1 for lightening skin, wherein the composition is administered such that the daily dosage for oral use of ketoconazole is 50 to 200, preferably 50 to 100 mg; and the daily dosage for oral use of sulforaphane is 50 to 600, preferably 200 to 400 mg.

8. A method of lightening the skin of a human, the method comprising the step of a person in need thereof imbibing a composition according to claim 1 such that the daily dosage of ketoconazole is 50 to 200, preferably 50 to 100 mg; and the daily dosage of sulforaphane is 50 to 600, preferably 200 to 400 mg.
